(19) United States
(12) Patent Application Publication
(10) Pub. No.: US 2025/0339651 A1
(43) Pub. Date: Nov. 6, 2025

(54) MEDICAL INSTRUMENT

(71) Applicant: ASAHI INTECC CO., LTD., Seto-shi (JP)

(72) Inventors: Ryuuya SHIRAISHI, Seto-shi (JP); Yukihiro FUSEYA, Seto-shi (JP); Ryo HOSOMI, Seto-shi (JP); Daisuke KURITA, Seto-shi (JP); Erina MINAMI, Seto-shi (JP)

(73) Assignee: ASAHI INTECC CO., LTD., Seto-shi (JP)

(21) Appl. No.: 19/272,692

(22) Filed: Jul. 17, 2025

Related U.S. Application Data
(63) Continuation of application No. PCT/JP2023/004193, filed on Feb. 8, 2023.

(51) Int. Cl. A61M 25/06; A61M 29/02

(52) U.S. Cl. CPC A61M 25/0662; A61M 29/02

(57) ABSTRACT

A medical instrument includes a shaft portion and a tip portion located at one end of the shaft portion and having an outer diameter that decreases in a direction away from the shaft portion. The shaft portion and the tip portion comprise a coil body including multiple wires wound in a hollow spiral shape. The coil body includes a first coil portion corresponding to the tip portion and a second coil portion corresponding to the shaft portion. The first coil portion is formed by thinning out at least one of the multiple wires.

This application is a bypass continuation of PCT/JP2023/004193 filed Feb. 8, 2023, the entire contents of the prior application being incorporated herein by reference.

### TECHNICAL FIELD

The disclosure relates to a medical instrument.

### BACKGROUND ART

Conventionally, for example, a dilator is known as a medical instrument. The dilator usually includes a tube portion having a hollow portion through which a guidewire is inserted, and a tapered portion disposed at the tip side of the tube portion (see, e.g., Patent literature 1). During the procedure, the tip end of an endoscope is first positioned near the site where a puncture hole is to be opened, and a puncture needle is advanced from the tip end of the endoscope to open the puncture hole. After that, the guidewire is inserted into the puncture hole through a lumen of the puncture needle, and then the puncture needle is removed. Next, a dilator is advanced along the guide wire to the puncture hole. The puncture hole is expanded by pushing the dilator into the puncture hole.

### CITATION LIST

### Patent Literature

Patent Literature 1: JP 2017-51328 A

### SUMMARY

### Technical Problem

A conventional dilator such as the one described in Patent literature 1 has room for improvement in terms of propulsion performance and removal performance in a puncture hole or a stenosed part.

The disclosure has been made in consideration of the above circumstances, and has an object to provide a medical instrument with improved propulsion performance and removal performance in a puncture hole or a stenosed part.

### Solution to Problem

In order to achieve the above object, the disclosure provides a medical instrument including a shaft portion and a tip portion located at one end of the shaft portion and having an outer diameter that decreases in a direction away from the shaft portion, the shaft portion and the tip portion being composed of a coil body having multiple wires wound in a hollow spiral shape, the coil body including a first coil portion corresponding to the tip portion and a second coil portion corresponding to the shaft portion, the first coil portion being formed by thinning out at least one of the multiple wires.

The coil body (multi-stranded coil) in which the multiple wires (wire materials) are closely arranged and wound acts like a screw on an inner wall (wall of an organ or the like) of the puncture hole or the stenosed part. Thus, the embodiment has superior propulsion performance inside the puncture hole or the stenosed part compared to a tube used in a conventional dilator. In addition, in this embodiment, the first coil portion corresponding to the tip portion is formed by thinning out at least one of the multiple wires. Thus, when the coil body is inserted in the puncture hole or the stenosed part, applying a twisting force in the insertion direction widens a gap between the wires at the tip portion and expands the outer diameter of the tip portion, thereby pushing open the inner wall of the puncture hole or the stenosed part. Further, in the first coil portion corresponding to the tip portion, a part corresponding to the thinned wire forms a spiral groove, facilitating the bite of the inner wall of the puncture hole or the stenosed part into this spiral groove. This promotes the rotation of the tip portion, making it easier to insert the coil body into the puncture hole or the stenosed part. When the coil body is removed from the puncture hole or the stenosed part, applying the twisting force in the removal direction narrows the gap between the wires at the tip portion and reduces the outer diameter of the tip portion, thereby weakening the bite of the inner wall of the puncture hole or the stenosed part into the spiral groove. This makes it easier to remove the coil body from the puncture hole or the stenosed part. In this manner, this embodiment has improved propulsion performance and removal performance in the puncture hole or the stenosed part.

In the above embodiment, the second coil portion may be formed by thinning out at least one of the multiple wires.

In this embodiment, both the first coil portion corresponding to the tip portion and the second coil portion corresponding to the shaft portion are formed by thinning out at least one of the multiple wires, so that the spiral grooves are present throughout the entire medical instrument and the number of spiral grooves is increased. Thus, when the medical instrument is inserted into the puncture hole or the stenosed part, applying the twisting force in the insertion direction expands the outer diameter of the entire medical instrument and allows the bite of the inner wall of the puncture hole or the stenosed part into the increased spiral grooves, making it easier to insert the medical instrument into the puncture hole or the stenosed part. When the medical instrument is removed from the puncture hole or the stenosed part, applying the twisting force in the removal direction reduces the outer diameter of the entire medical instrument and weakens the bite of the inner wall of the puncture hole or the stenosed part into the spiral grooves, making it easier to remove the medical instrument from the puncture hole or the stenosed part. In this manner, the embodiment has further improved propulsion performance and removal performance in the puncture hole or the stenosed part.

In the above embodiment, when the number of the wires thinned out in the first coil portion is defined as N₁ and the number of the wires thinned out in the second coil portion is defined as N₂, N₁>N₂ may be satisfied.

According to this embodiment, the flexibility of the tip portion is higher than that of the shaft portion, improving propulsion performance of the tip portion in the puncture hole or the stenosed part. Further, the rigidity of the shaft portion is higher than that of the tip portion, making it possible to prevent a decrease in torque transmission.

In the above embodiments, the coil body may be a single coil body.

According to this embodiment, the twisting force applied when the medical instrument is inserted into the puncture hole or the stenosed part and the twisting force applied when the medical instrument is removed from the puncture hole or the stenosed part are easily transmitted to the tip portion. Thus, propulsion performance and removal performance in the puncture hole or the stenosed part are further improved.

In the above embodiments, when the number of the multiple wires is defined as N and the number of the wires thinned out in the first coil portion is defined as N₁, N₁/N may satisfy a value of 0.4 or less.

According to this embodiment, it is possible to prevent the rigidity of the tip portion from excessively decreasing.

In the above embodiment, the coil body may be a single coil body and may be formed by thinning out at least one of the multiple wires.

According to this embodiment, the coil body is formed by thinning out at least one of the multiple wires, so that the number of the spiral grooves is increased. Thus, propulsion performance and removal performance in the puncture hole or the stenosed part are improved.

In the above embodiment, when the number of the multiple wires is defined as N and the number of the wires thinned out in the coil body is defined as N_{c}, N_{c}/N may satisfy a value of 0.4 or less.

According to this embodiment, it is possible to prevent the rigidity of the tip portion and the shaft portion from excessively decreasing.

In the above embodiments, the coil body may be the single coil body, and the diameter of the multiple wires may gradually decrease from the second coil portion toward the first coil portion.

According to this embodiment, the flexibility of the tip portion is higher than that of the shaft portion, improving propulsion performance of the tip portion in the puncture hole or the stenosed part. Further, the rigidity of the shaft portion is higher than that of the tip portion, making it possible to prevent a decrease in torque transmission.

### Advantageous Effects

According to the disclosure, it is possible to provide the medical instrument having improved propulsion performance and removal performance in the puncture hole or the stenosed part.

### BRIEF DESCRIPTION OF DRAWINGS

FIG. 1 is a schematic side view illustrating a structure of a medical instrument according to one embodiment.

FIG. 2A is a partial side perspective view illustrating a tip portion of the medical instrument in FIG. 1.

FIG. 2B is a cross-sectional view of IIA-IIA in FIG. 2A.

FIG. 2C is a partial side perspective view illustrating a shaft portion of the medical instrument in FIG. 1.

FIG. 2D is a cross-sectional view of IIC-IIC in FIG. 2C.

FIG. 3 is a schematic side view describing an operation in which the medical instrument in FIG. 1 is inserted into a puncture hole.

FIG. 4A is a partial cross-sectional view describing the operation in which the medical instrument in FIG. 1 is inserted into the puncture hole.

FIG. 4B is a partial cross-sectional view describing an operation in which the medical instrument in FIG. 1 is removed from the puncture hole.

FIG. 5A is a partial side perspective view illustrating the tip portion of the medical instrument of a modification 1.

FIG. 5B is a cross-sectional view of VA-VA in FIG. 5A.

FIG. 5C is a partial side perspective view illustrating the shaft portion of the medical instrument of the modification 1.

FIG. 5D is a cross-sectional view of VC-VC in FIG. 5C.

FIG. 6 is a schematic side view illustrating the structure of the medical instrument of a modification 2.

### DETAILED DESCRIPTION OF EMBODIMENTS

Hereinafter, embodiments are described based on the drawings. The disclosure is not limited to the embodiments described below, and the described embodiments are merely examples for explaining the technical features of the disclosure. Further, the shapes and dimensions illustrated in the drawings are shown only to facilitate understanding of the contents of the disclosure, and do not accurately reflect the actual shapes and dimensions.

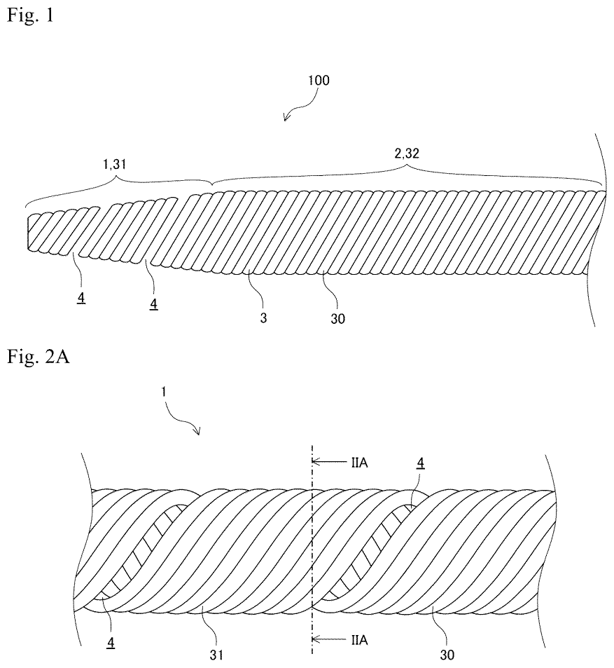

FIG. 1 is a schematic side view illustrating a structure of a medical instrument 100 according to an embodiment. In the present embodiment, the medical instrument 100 is a dilator. The medical instrument 100 has an elongated shape. As illustrated in FIG. 1, the medical instrument 100 includes a shaft portion 2 and a tip portion 1 which is located at a tip end of the shaft portion 2 and has an outer diameter that decreases in a direction away from the shaft portion 2 (direction toward the tip end).

In the present specification, the “tip side” refers to a direction along the axial direction of the medical instrument 100, that is, the direction in which the medical instrument 100 advances toward the puncture hole or the stenosed part. The “base side” refers to a direction along the axial direction of the medical instrument 100, that is, the direction opposite to the tip side described above. Further, the “tip end” refers to an end portion of any member or site on the tip side, and the “base end” refers to an end portion of any member or site on the base side. Note that, in FIG. 1, the left side of the drawing is the “tip side” which is inserted into the puncture hole or the stenosed part, and the right side of the drawing is the “base side” which is operated by an operator.

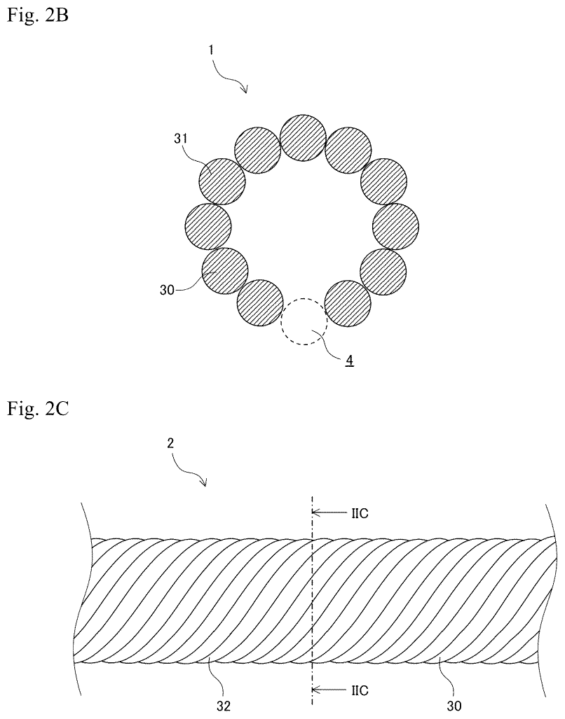

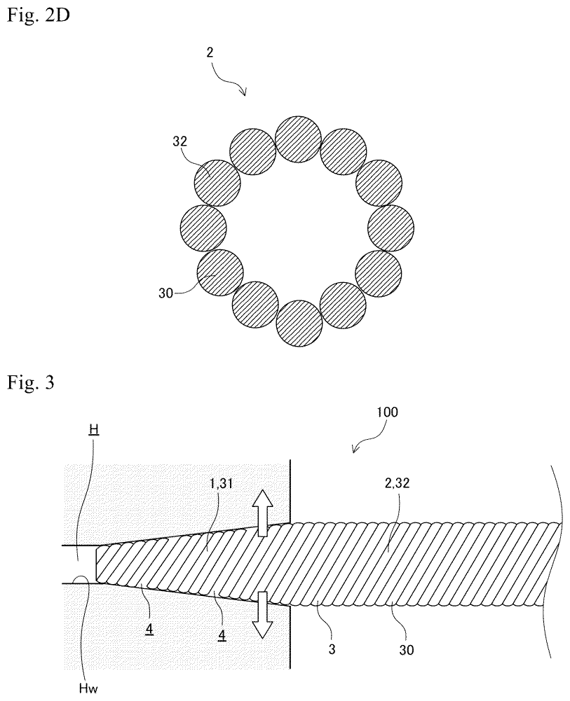

FIG. 2A is a partial side perspective view illustrating the tip portion 1 of the medical instrument 100, and FIG. 2B is a cross-sectional view of IIA-IIA in FIG. 2A. FIG. 2C is a partial side perspective view illustrating the shaft portion 2 of the medical instrument 100, and FIG. 2D is a cross-sectional view of IIC-IIC in FIG. 2C. As illustrated in FIG. 1, the shaft portion 2 and the tip portion 1 comprise a coil body 3 having multiple wires 30 wound in a hollow spiral shape. As illustrated in FIG. 1 and FIG. 2A to FIG. 2D, the coil body 3 includes a first coil portion 31 corresponding to the tip portion 1 and a second coil portion 32 corresponding to the shaft portion 2. In the medical instrument 100 of the present embodiment, the first coil portion 31 is formed by thinning out at least one of the multiple wires 30.

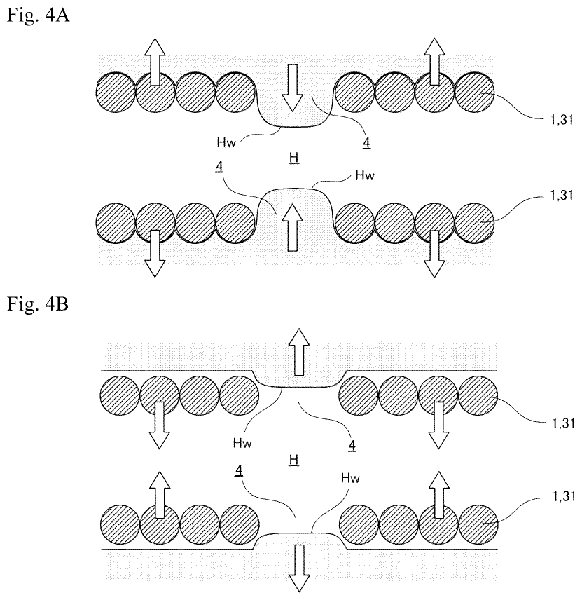

FIG. 3 is a schematic side view for explaining an operation in which the medical instrument 100 is inserted into a puncture or a stenosed part (hereinafter simply referred to as “hole H”). FIG. 3 is a schematic side view describing an operation in which the medical instrument in FIG. 1 is inserted into a puncture hole. FIG. 4A is a partial cross-sectional view for explaining the operation in which the medical instrument 100 is inserted into the hole H, and FIG. 4B is a partial cross-sectional view for explaining an operation in which the medical instrument 100 is removed from the hole H.

As illustrated in FIG. 3, the coil body (multi-strand coil) 3, which is formed by closely arranging and winding the multiple wires (wire materials) 30, acts like a screw on an inner wall (wall of an internal organ) Hw of the hole H, and thus has superior propulsion performance inside the hole H compared to a tube used in a conventional dilator. In addition, as illustrated in FIG. 1, and FIG. 2A and FIG. 2B, in the medical instrument 100 of the present embodiment, the first coil portion 31 corresponding to the tip portion 1 is formed by thinning out at least one of the multiple wires 30. Thus, when the medical instrument is inserted into the hole H, applying a twisting force in the insertion direction widens a gap between the wires 30 at the tip portion 1 and expands the outer diameter of the tip portion 1, thereby pushing open the inner wall Hw of the hole H. Further, as illustrated in FIG. 4A, in the first coil portion 31 corresponding to the tip portion 1, a part corresponding to the thinned wire 30 forms a spiral groove 4, facilitating the bite of the inner wall Hw of the hole H into the spiral groove 4. This promotes the rotation of the tip portion 1, making it easier to insert the medical instrument 100 into the hole H. As illustrated in FIG. 4B, when the medical instrument is removed from the hole H, applying the twisting force in the removal direction narrows the gap between the wires 30 at the tip portion 1 and reduces the outer diameter of the tip portion 1, thereby weakening the bite of the inner wall Hw of the hole H into the spiral groove 4. This makes it easier to remove the medical instrument 100 from the hole H. In this manner, the medical instrument 100 of the present embodiment has improved propulsion performance and removal performance in the hole H.

When the medical instrument 100 is inserted into the hole H, the rotation direction (twist direction) of the medical instrument 100 is preferably the same phase as the winding direction of the multiple wires 30 in the coil body 3. For example, if the rotation direction (twist direction) of the medical instrument 100 when it is inserted into the hole H is clockwise, the winding direction of the multiple wires 30 in the coil body 3 is preferably clockwise. With this configuration, applying the twisting force in the insertion direction widens the gap between the wires 30 at the tip portion 1 and expands the outer diameter of the tip portion 1.

In the present embodiment, the coil body 3 is a single coil body. That is, the first coil portion 31 corresponding to the tip portion 1 and the second coil portion 31 corresponding to the shaft portion 2 are continuous. With this configuration, the twisting force is easily transmitted to the tip portion 1 when the medical instrument 100 is inserted into the hole H and when it is removed from the hole H. Thus, propulsion performance and removal performance of the medical instrument 100 in the hole H are further improved.

The number of the wires 30 constituting the coil body 3 is not particularly limited. The number of the wires 30 may be, for example, 12. FIG. 2A and FIG. 2B illustrate an example of the first coil portion 31 formed by thinning out one of the 12 wires 30. In FIG. 2A and FIG. 2B, the thinned wire 30 is shown by dashed lines. FIG. 2C and FIG. 2D illustrate an example of the second coil portion 32 formed by the 12 wires 30. In FIG. 2A and FIG. 2B, the thinned wire 30 is shown by dashed lines.

The number of the wires 30 thinned out in the first coil portion 31 is not particularly limited. However, when the number of the multiple wires 30 is defined as N and the number of the wires 30 thinned out in the first coil portion 31 is defined as N₁, N₁/N preferably satisfies a value of 0.4 or less. For example, when the number N of the multiple wires 30 is 12, the number N₁ of the wires 30 thinned out in the first coil portion 31 is preferably 1 to 4. With this configuration, it is possible to prevent the rigidity of the tip portion 1 from excessively decreasing.

When the number N₁ of the wires 30 thinned out in the first coil portion 31 is 2 or more, these thinned wires 30 may or may not be adjacent to each other. For example, when the number N of the multiple wires 30 is 12 and the number N₁ of the wires 30 thinned out in the first coil portion 31 is 2, the thinned wires 30 may be the 2 adjacent wires 30, or may be a total of the 2 wires 30 which are thinned out so that there is another wire 30 between them.

In the medical instrument 100, an end portion of the wires 30 may be present at a boundary between the second coil portion 32 and the first coil portion 31.

Examples of the wire 30 include a solid wire material and a twisted wire material having a structure in which multiple twisted wires are twisted together. In general, the rigidity of the solid wire material is higher than that of the twisted wire material. When the wire 30 is the solid wire material, the diameter may be, for example, about 0.2 mm. When the wire 30 is the twisted wire material, the twisted wire material may be formed, for example, by twisting together the 5 to 8 twisted wires with a diameter of about 0.05 mm.

The medical instrument 100 of the present embodiment can be produced, for example, by the following method. First, the multiple wires 30 are wound around a core material including an elongated portion having a constant outer diameter corresponding to the second coil portion 32 (shaft portion 2) and a cone-shaped portion having an outer diameter that decreases toward the tip end corresponding to the first coil portion 31 (tip portion 1). In this case, at least one of the multiple wires 30 is thinned out in a part corresponding to the first coil portion 31 to form the hollow spiral coil body 3. In this manner, the medical instrument 100 is obtained.

### Modification 1

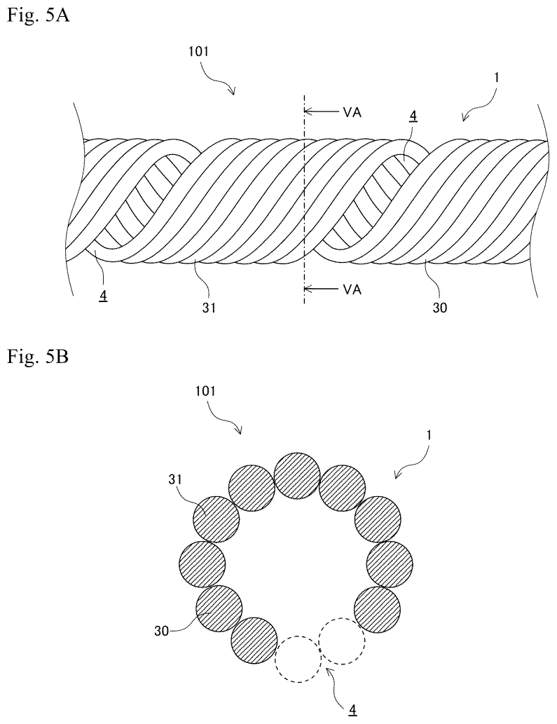

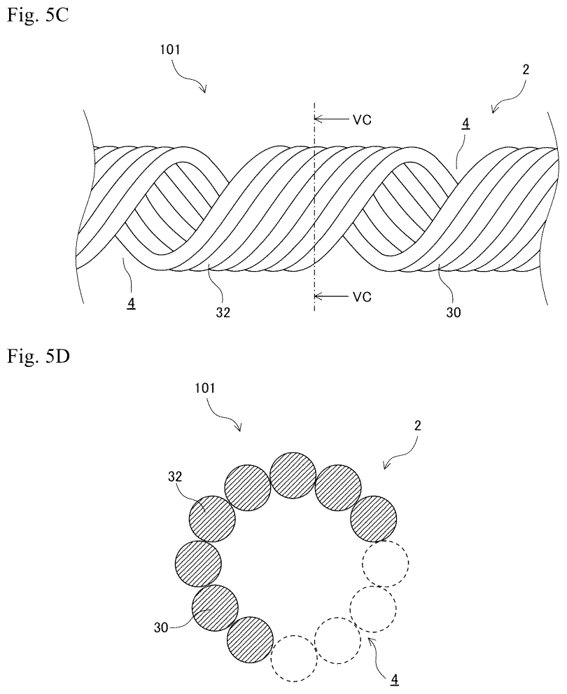

FIG. 5A is a partial side perspective view illustrating the tip portion 1 of a medical instrument 101 of a modification 1, and FIG. 5B is a cross-sectional view of VA-VA in FIG. 5A. FIG. 5C is a partial side perspective view illustrating the shaft portion 21 of the medical instrument 101 of the modification 1, and FIG. 5D is a cross-sectional view of VC-VC in FIG. 5C. The medical instrument 101 of the modification 1 has the same configuration as the above-mentioned medical instrument 100, except that, in addition to the first coil portion 31 corresponding to the tip portion 1, the second coil portion 32 corresponding to the shaft portion 2 is also formed by thinning out at least one of the multiple wires 30.

In the medical instrument 101, both the first coil portion 31 corresponding to the tip portion 1 and the second coil portion 32 corresponding to the shaft portion 2 are formed by thinning out at least one of the multiple wires 30, so that the spiral grooves 4 are present throughout the entire medical instrument 101, and the number of the spiral grooves 4 is increased. Thus, when the medical instrument 101 is inserted into the hole H, applying the twisting force in the insertion direction expands the outer diameter of the entire medical instrument 101 and allows the bite of the inner wall Hw of the hole H into the increased spiral grooves 4, making it easier to insert the medical instrument 101 into the hole H. When the medical instrument 101 is removed from the hole H, applying the twisting force in the removal direction reduces the outer diameter of the entire medical instrument 101 and weakens the bite of the inner wall Hw of the hole H into the spiral grooves 4, making it easier to remove the medical instrument 101 from the hole H. In this manner, the medical instrument 101 of the modification 1 has further improved propulsion performance and removal performance of the medical instrument 101 in the hole H.

In the second coil portion 32, the number of the thinned wires 30 is not particularly limited. However, when the number of the wires 30 thinned out in the second coil portion 32 is defined as N₂, it is preferable to satisfy N₁>N₂. That is, it is preferable that the number N₁ of the wires 30 thinned out in the first coil portion 31 is greater than the number N₂ of the wires 30 thinned out in the second coil portion 32. With this configuration, the flexibility of the tip portion 1 is higher than that of the shaft portion 2, and as a result, propulsion performance of the tip portion 1 in the hole H is improved. Further, the rigidity of the shaft portion 2 is higher than that of the tip portion 1, and as a result, a decrease in torque transmission can be prevented. For example, when the number N of the multiple wires 30 is 12, the number N₁ of the wires 30 thinned out in first coil portion 31 may be 3 or 4, and the number N₂ of the wires 30 thinned out in second coil portion 32 may be 1 or 2.

When the number N₁ of the wires 30 thinned out in the first coil portion 31 is greater than the number N₂ of the wires 30 thinned out in the second coil portion 32, in the medical instrument 101, an end portion of the wires 30 may be present at the boundary between the second coil portion 32 and the first coil portion 31.

Although not illustrated, the number N₁ of the wires 30 thinned out in the first coil portion 31 may be equal to the number N₂ of the wires 30 thinned out in the second coil portion 32. In this case, the coil body 3, which is a single coil body, is formed by thinning out at least one of the multiple wires 30. This configuration also improves propulsion performance and removal performance of the medical instrument 101 in the hole H. Further, since there is no need to change the number of the wires 30 to be thinned out between the first coil portion 31 and the second coil portion 32, the medical instrument 101 can be easily produced.

When the number N₁ of the wires 30 thinned out in the first coil portion 31 is equal to the number N₂ of the wires 30 thinned out in the second coil portion 32, the number of the wires 30 thinned out in the coil body 3 is not particularly limited. However, when the number of the wires 30 thinned out in the coil body 3 is defined as N_{c}, N_{c}/N preferably satisfies a value of 0.4 or less. With this configuration, it is possible to prevent the rigidity of the medical instrument 101 from excessively decreasing. For example, when the number N of the multiple wires 30 is 12, the number N_{c} of the wires 30 thinned out in the coil body 3 may be 1 to 4.

FIG. 5A and FIG. 5B illustrate an example of the first coil portion 31 formed by thinning out 2 adjacent wires out of the 12 wires 30. FIG. 5C and FIG. 5D illustrate an example of the second coil portion 32 formed by thinning out 4 adjacent wires out of the 12 wires 30. In FIG. 5A to FIG. 5D, the thinned wires 30 are shown by dashed lines.

When the number N₂ of the wires 30 thinned out in the second coil portion 32 is 2 or more, these thinned wires 30 may be adjacent or not be adjacent to each other. For example, when the number N of the multiple wires 30 is 12 and the number N₂ of the wires 30 thinned out in the second coil portion 32 is 2, the wires 30 thinned out in the second coil portion 32 may be the two adjacent wires 30, or may be a total of the two wires 30 which are thinned out so that there is another wire 30 between them.

The medical instrument 101 of the modification 1 can be produced, for example, by the following method. First, the multiple wires 30 are wound around a core material including an elongated portion having a constant outer diameter corresponding to the second coil portion 32 (shaft portion 2) and a cone-shaped portion having an outer diameter that decreases toward the tip end corresponding to the twelfth coil portion 31 (tip portion 1). In this case, at least one of the multiple wires 30 is thinned out in a part corresponding to the first coil portion 31 and a part corresponding to the second coil portion 32 to form the hollow spiral coil body 3. In this manner, the medical instrument 101 is obtained.

### Modification 2

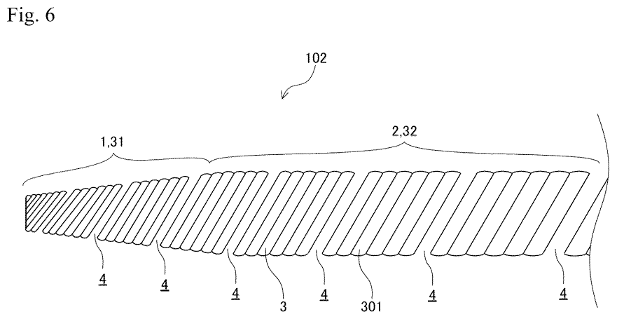

FIG. 6 is a schematic side view illustrating a structure of a medical instrument 102 of a modification 2. The medical instrument 102 of the modification 2 has the same configuration as the above-mentioned medical instruments 100 and 101, except that the diameter of the multiple wires 301 constituting the coil body 3 gradually decreases from the second coil portion 32 toward the first coil portion 31.

According to the medical instrument 102 of the modification 2, the flexibility of the tip portion 1 is higher than that of the shaft portion 2, improving propulsion performance of the tip portion 1 in the hole H. Further, the rigidity of the shaft portion 2 is higher than that of the tip portion 1, making it possible to prevent a decrease in torque transmission.

As the wires 301, for example, the wire materials described for the wires 30 can be used.

When the diameter of the wires 301 at the base portion of the second coil portion 32 is defined as R₂ and the diameter of the wires 301 at the tip portion of the first coil portion 31 is defined as R₁, the wires 301 may satisfy 0.1≤R₁/R₂≤0.7. With this configuration, it is possible to achieve both the flexibility of the tip portion 1 and the rigidity of the shaft portion 2.

FIG. 6 illustrates an example of the medical instrument 102 comprising the coil body 3 that is a single coil body formed by thinning out one of the multiple wires 301. The medical instrument 102 is not limited to the example illustrated in FIG. 6. In the medical instrument 102, only the first coil portion 31 corresponding to the tip portion 1 may be formed by thinning out at least one of the multiple wires 301. In the medical instrument 102, both the first coil portion 31 corresponding to the tip portion 1 and the second coil portion 32 corresponding to the shaft portion 2 may be formed by thinning out at least one of the multiple wires 301, and the number of the wires 301 thinned out in the first coil portion 31 may be equal to or different from the number of the wires 301 thinned out in the second coil portion 32. The number of the wires 301 thinned out in the first coil portion 31 may be greater than the number of the wires 301 thinned out in second coil portion 32.

In the medical instrument 102 of the modification 2, the shaft portion 2 may have the outer diameter that decreases toward the tip end. Such a medical instrument 102 can be produced, for example, by the following method. First, the multiple wires 301 are wound around a core material including an elongated portion having a constant outer diameter corresponding to the second coil portion 32 (shaft portion 2) and a cone-shaped portion having an outer diameter that decreases toward the tip end corresponding to the twelfth coil portion 31 (tip portion 1), so that the diameter of the wires 301 decreases from the second coil portion 32 toward the first coil portion 31. In this case, at least one of the multiple wires 301 is thinned out in the part corresponding to the first coil portion 31 (and, if necessary, the part corresponding to the second coil portion 32) to form the hollow spiral coil body 3. In this manner, the medical instrument 102 having the shaft portion 2 whose outer diameter decreases toward the tip end is obtained.

In the medical instrument 102 of the modification 2, the shaft portion 2 may have an inner diameter that increases toward the tip end. Such a medical instrument 102 can be produced, for example, by the following method. First, the above-mentioned multiple wires 30 are wound around a core material including an elongated portion having a constant outer diameter corresponding to the second coil portion 32 (shaft portion 2) and a cone-shaped portion having an outer diameter that decreases toward the tip end corresponding to the first coil portion 31 (tip portion 1). In this case, at least one of the multiple wires 30 is thinned out in the part corresponding to the first coil portion 31 (and, if necessary, the part corresponding to the second coil portion 32) to form a hollow spiral coil body. Next, the resulting coil body is subjected to a chemical treatment, polishing, or the like to gradually reduce the diameter of the wires 30 from the second coil portion 32 toward the first coil portion 31. That is, the wires 30 are converted to the wires 301. In this manner, the medical instrument 102 having the shaft portion 2 whose inside diameter increases toward the tip end is obtained.

Although the preferable embodiments and modifications of the medical instrument according to the disclosure have been described above with reference to the drawings, the disclosure is not limited to the above-mentioned embodiments and modifications. Various modifications, substitutions, and the like can be applied to the above-mentioned embodiments and modifications without departing from the scope of the disclosure. Further, each of the features described with reference to the above-mentioned embodiments and modifications may be appropriately combined as long as there is no technical contradiction.

For example, in the above-mentioned embodiments, the disclosure is described using an example in which the medical instrument is used as a dilator. However, the medical instrument of the disclosure is not limited to the use as a dilator, and may be applied to, for example, a catheter such as a penetrating catheter or a guiding catheter.

1. A medical instrument comprising:
a shaft portion; and
a tip portion located at one end of the shaft portion and having an outer diameter that decreases in a direction away from the shaft portion, wherein
the shaft portion and the tip portion comprise a coil body including multiple wires wound in a hollow spiral shape,
the coil body includes a first coil portion corresponding to the tip portion and a second coil portion corresponding to the shaft portion, and
the first coil portion includes at least one of the multiple wires that is thinned out

2. The medical instrument according to claim 1, wherein the second coil portion includes at least one of the multiple wires that is thinned out.

3. The medical instrument according to claim 2, wherein when the number of the wires thinned out in the first coil portion is defined as N₁ and the number of the wires thinned out in the second coil portion is defined as N₂, the medical instrument satisfies N₁>N₂.

4. The medical instrument according to claim 1, wherein the first coil portion and the second coil portion are formed of a single coil body.

5. The medical instrument according to claim 1, wherein when the number of the multiple wires is defined as N and the number of the wires thinned out in the first coil portion is defined as N₁, N₁/N satisfies a value of 0.4 or less.

6. The medical instrument according to claim 1, wherein the first coil portion and the second coil portion are formed of a single coil body including at least one of the multiple wires that is thinned out.

7. The medical instrument according to claim 6, wherein when the number of the multiple wires is defined as N and the number of the wires thinned out in the coil body is defined as N_{C}, N_{C}/N satisfies a value of 0.4 or less.

8. The medical instrument according to claim 1, wherein the first coil portion and the second coil portion are formed of a single coil body, and the multiple wires gradually decrease in diameter from the second coil portion toward the first coil portion.

9. The medical instrument according to claim 1, wherein a part corresponding to the at least one of the multiple wires that is thinned out in the first coil portion is a spiral groove.

10. The medical instrument according to claim 2, wherein a part corresponding to the at least one of the multiple wires that is thinned out in the second coil portion is a spiral groove.

11. The medical instrument according to claim 10, wherein a part corresponding to the at least one of the multiple wires that is thinned out in the first coil portion is a spiral groove.
